(19) United States
(12) Patent Application Publication
(10) Pub. No.: US 2008/0202513 A1
(43) Pub. Date: Aug. 28, 2008

(54) Use of Dry Powder Compositions For Pulmonary Delivery

(76) Inventors: James Caradoc Birchall, Cardiff (GB); Haoying Li, Cardiff (GB)

Correspondence Address: FOX ROTHSCHILD LLP;PRINCETON PIKE CORPORATE CENTER, 997 LENOX DRIVE, BUILDING #3, LAWRENCEVILLE, NJ, 08648, US

(21) Appl. No.: 11/632,938

(22) PCT Filed: Jul. 20, 2005
(86) PCT No.: PCT/GB05/02838

§ 371 Date: Dec. 18, 2007

(30) Foreign Application Priority Data
Jul. 21, 2004 (GB) 0416328.3

(51) Int. Cl. A61K 9/14; A61K 31/722; A61M 15/00

(52) U.S. Cl. 128/203.15; 424/489; 514/55

(57) ABSTRACT

Use of chitosan or a chitosan derivative in a dry powder composition enhances the release of the dry powder composition containing a medicament from a container and/or the dispersibility in air of the dry powder composition containing a medicament. When the dry powder composition is administered to a person in need thereof by means of a dry powder inhaler, the presence of the chitosan or chitosan derivative enhances the dispersibility of the dry powder composition such that the dose emitted from the container is enhanced and/or the respirable fraction available for deposit in the lungs of a person is increased.

The present invention relates inter alia to the use of dry powder compositions suitable for pulmonary delivery, to a dry powder composition per se, to containers, adapted for use with dry powder inhalers, containing dry powder compositions and to dry powder inhalers incorporating such containers.

WO 96/05810 describes the use of chitosan particles to improve the absorption of drugs across mucosal tissue. Compositions comprising chitosan are said to be administered inter alia via the nasal route as a powder using a nasal powder device and via the pulmonary route using a powder inhaler or metered dose inhaler.

WO 98/01160 describes a composition comprising a particulate complex of chitosan and DNA wherein the complex is between 10 nm and 1 μm in size and carries a surface charge. The chitosan complexes are used as non-viral vectors to enhance the expression of nucleic acid in epithelial tissues such as inter alia the nasal cavity, the lungs and the buccal cavity. Aerosol systems such as propellant devices, dry powder systems and nebulizers can be used for administration to the lungs.

G. Borchard, Advanced Drug Delivery Reviews 52 (2001) 145-150 is a review article that examines the advances in the application of chitosan and chitosan derivatives to non-viral gene delivery.

WO 01/78689 describes the use of chitosan as a matrix or carrier in a particulate composition containing a medicament, the particulate composition being suitable for use in an aerosol composition including a liquid propellant.

The object of the present invention is to enhance the release from a container adapted for use with, for example, a dry powder inhaler and/or the dispersibility in air of particulate medicaments intended for, for example, pulmonary delivery employing, for example, a dry powder inhaler.

Throughout the present specification by “medicament” is meant any pharmacologically active material, including nucleic acids. “Nucleic acids” includes nucleic acids such as DNA, oligonucleotides greater than 100 base pairs, plasmids or cosmids thereof, coiled or uncoiled.

Throughout the present specification, unless otherwise indicated, by “air” is meant any ambient gaseous phase, including the ambient gaseous phase within a patient's upper and lower airways.

According to a first aspect of the present invention there is provided use of chitosan or a derivative thereof as a means for enhancing the release from a container and/or for improving the dispersibility in air of a dry powder composition containing a medicament.

According to a second aspect of the present invention there is provided use of chitosan or a derivative thereof in the manufacture of a dry powder composition containing a medicament for improving the delivery of the medicament to the lungs in a patient in need thereof. The improvement can comprise the enhancement of the release from a container of the dry powder composition and/or better dispersibility in air of the dry powder composition.

Suitably the present invention uses a dry powder composition which comprises the medicament and the chitosan or chitosan derivative and which is administered from a container by means of a dry powder inhaler so as to deliver at least the medicament to the lungs of a patient in need thereof.

According to a third aspect of the present invention there is provided a method for delivering a medicament to the lungs of a patient in need thereof comprising administering the medicament in the form of a dry powder composition, the dry powder composition containing additionally chitosan or a derivative thereof, the chitosan or derivative thereof being present to enhance the release from a container and to improve the dispersibility in air of the dry powder composition.

According to a fourth aspect of the present invention there is provided a container adapted for use with a dry powder inhaler, the container containing a dry powder composition comprising a particulate medicament and a chitosan derivative, the chitosan derivative comprising chitosan substituted at, at least some of, its NH₂ sites by one, two or three members selected from the group comprising C₁ to C₆ alkyl and C₁ to C₆ acyl.

According to a fifth aspect of the present invention there is provided a container adapted for use with a dry powder inhaler, the container containing a dry powder composition comprising a particulate medicament and chitosan or a chitosan derivative, wherein the chitosan or chitosan derivative is water soluble and/or contains no cross-linking formed with a cross linking agent and comprises particles having an average diameter greater than 1 μm.

According to a sixth aspect of the present invention there is provided a container adapted for use with a dry powder inhaler, the container containing a dry powder composition comprising a particulate medicament and chitosan or a chitosan derivative, wherein the chitosan or chitosan derivative comprises particles having an average diameter of more than 3.5 μm.

According to a seventh aspect of the present invention there is provided a dry powder composition comprising 0.001 to 30 wt % medicament, 1 to 50 wt %, preferably 1 to 40 wt %, chitosan or a chitosan derivative and 49.999 to 98.999 wt %, preferably 59.999 to 98.999 wt %, disaccharide, in which particulate material comprising at least the medicament comprises particles having an average diameter of from 0.5 to 11 μm, preferably 0.5 to 3.5 μm.

According to an eighth aspect of the present invention there is provided a container adapted for use with a dry powder inhaler, the container containing a dry powder composition according to the seventh aspect of the invention.

According to a further aspect of the present invention there is provided a dry powder inhaler adapted for administering a particulate medicament or medicament-containing particles to the lungs of a patient in need thereof, the dry powder inhaler incorporating a container containing the dry powder composition comprising the particulate medicament or medicament-containing particles and a chitosan or chitosan derivative, the container being according to any of the fourth, fifth, sixth or eighth aspects of the present invention.

Use of chitosan or a chitosan derivative according to the present invention has been found to enhance the release of a dry powder composition from a container and/or to improve the dispersibility in air of a dry powder composition.

Preferred means for carrying out the present invention are set out in dependent claims 2 to 14.

Any of the above fourth, fifth, sixth, seventh, eighth or further aspects of the invention can be used in combination with any of the above first, second and third aspects of the invention.

Dispersibility of a dry powder composition is important at a number of stages in the use of, for example, a dry powder inhaler employed to deliver a particulate medicament or medicament-containing particles contained in the dry powder composition to the lungs of a patient in need thereof. Dispersibility of the dry powder composition is required, firstly, to release the dry powder composition from a container within the dry powder inhaler, secondly, if necessary, to separate the emitted fraction into a respirable and a non-respirable fraction and, thirdly, if necessary, to separate the respirable fraction into a fraction that deposits in the upper airways and a fraction that is capable of being deposited in the lower airways of a patient to which the dry powder composition is administered. The present invention has been found to be capable of enhancing the dispersibility of a dry powder composition at each of these stages.

A dry powder inhaler employs a container in the form of, for example, a capsule, blister pack or reservoir, containing a dry powder composition. A known problem is, on activating the inhaler, the retention of a portion of the dry powder composition either within individual dosage containers such as a capsule or a blister pack inserted into the dry powder inhaler or in the reservoir or the dosage mechanism of a metered dose dry powder inhaler employing bulk storage of the dry powder composition.

Use of the present invention can permit at least 70 wt % of an intended dose, such as that, for example, contained within an individual capsule, to be emitted from the dry powder inhaler. Suitably at least 80 wt %, even more suitably at least 90 wt %, measured with respect to the total weight of the intended dose, for example the contents of one capsule, to be emitted. Higher levels of emitted dose are achieved as the relative amount of chitosan or chitosan derivative present in the dry powder composition is increased.

Use of the present invention with, for example, a dry powder inhaler can yield a separable fraction of at least 30 wt %, preferably at least 40 wt %, more preferably at least 50 wt %, measured with respect to the total weight of an intended dose of the dry powder composition, for example, such as the total contents of an individual capsule, the fraction comprising particles having an average diameter of not more than 11 μm.

Use of the present invention with, for example, a dry powder inhaler can yield a separable fraction of at least 5 wt %, preferably at least 10 wt %, more preferably at least 20 wt %, measured with respect to the total weight of an intended dose of the dry powder composition, for example, such as the total contents of an individual capsule, the said separable fraction comprising particles having an average diameter of not more than 3.5 μm.

Particulate materials having an average diameter within the range 0.5 to 11 μm are an appropriate size to enter the lungs. Particles having an average diameter of less than 0.05 μm are generally so small that if inhaled, they may not be trapped within the lung but may be exhaled. Particles having an average diameter of more than 13 μm are generally too large to enter the upper airways of the lungs. A fraction of dry powder composition comprising particles having an average diameter within the range 0.5 to 11 μm is thus deemed a respirable fraction.

To be deposited deep within the lungs, i.e. within the lower airways and at the mucosal surface of the alveoli, the particles produced by the dry powder inhaler must be sufficiently small to enter into the narrow lower airways of the lungs. Such particles should have an average diameter of not more than 3.5 μm, preferably within the range 0.5 to 3.5 μm, more preferably within the range 0.5 to 3.1 μm. Use of the present invention can thus provide a separable fraction of dry powder composition capable of being deposited within the lower airways of a patient.

A fraction of dry powder composition comprising particles having an average diameter within the range 3.5 μm to 11 μm is generally deposited in the upper and wider airways of a patient to which the dry powder composition is administered by, for example, means of a dry powder inhaler.

A fraction of dry powder composition comprising particles having an average diameter greater than 11 μm are generally deposited in the mouth and throat of a patient to which the dry powder composition is administered.

A medicament whose pharmaceutical action is intended to be delivered to and/or expressed in the lower airways, including the alveoli, of a patient needs to reach those airways and thus needs to be in the form of particles having an average diameter in the range of from 0.5 to 3.5 μm. A medicament whose pharmaceutical action is intended to be delivered to and/or expressed in the upper airways of a patient can have an average diameter in the range of from 3.5 to 11 μm. A medicament whose pharmaceutical action can be expressed in any part of the lung can have an average diameter of from 0.5 to 11 μm.

The chitosan or chitosan derivative employed will be selected having regard inter alia to the action that it is desired that the chitosan or chitosan derivative should deliver. Enhanced dispersibility of the medicament compound or composition containing a medicament will occur employing any chitosan or chitosan derivative, preferably a chitosan or chitosan derivative having a molecular weight within the range 5 kDa to 2000 kDa.

Suitably, however, the chitosan or derivative thereof in the dry powder composition comprises particles of chitosan or the derivative thereof having an average diameter within the range of from 0.5 to 200 μm.

Delivery of chitosan or a chitosan derivative to the alveoli of the lungs and hence to exhibit the property to enhance drug absorption through the alveoli, will only be shown by chitosan or a chitosan derivative able to reach the alveoli mucosal surface and, hence, by particles of chitosan or chitosan derivative having an average diameter of from 0.5 to 3.5 μm. A dry powder composition comprising particulate medicament and chitosan or chitosan derivative wherein the chitosan or chitosan derivative particles have an average diameter of more than 3.5 μm will not enhance the drug absorption through the alveoli mucosal surface as the particles of chitosan or chitosan derivative will be too large to pass through the lower airways to reach the alveoli.

A preferred particle size for the chitosan or chitosan derivatives in the dry powder composition is at least 1 μm and up to 5 μm. Such chitosan or chitosan derivatives can be prepared by, for example, spray drying, either with or without the medicament present in the spray drying mix. Where the medicament is not co-spray dried with the chitosan or chitosan derivative, it can, for example, be spray dried separately and then mixed with the spray dried chitosan or chitosan derivative.

An alternative preferred particle size for the chitosan or chitosan derivatives is at least 30 μm and up to 100 μm. Such chitosan or chitosan derivatives can, for example, be admixed with medicament that has, for example, been separately spray dried.

Throughout the specification by “average diameter” or “particle size” is meant the average diameter or particle size measured by laser diffraction.

The present invention permits the use of chitosan or a derivative of chitosan that contains no cross linking formed with a cross linking agent and hence is non-toxic. Use of a cross linking agent usually requires an initiator. Cross linking agents and initiators are highly reactive species. Due to their reactivity they are frequently toxic. Complete removal of residual reactants is not usually achievable. Use of a cross linking agent to form a cross linked chitosan or chitosan derivative that is a component of a pharmaceutical composition is thus not deemed desirable as the resultant product may include traces of toxic reactants.

Use of the present invention permits the use of a dry powder composition to be administered in use to the lungs of a patient by means of, for example, a dry powder inhaler, wherein the dry powder composition has, or medicament-containing particles in the dry powder composition have, been prepared by a process selected from the group comprising spray drying, freeze drying and mechanical techniques, such as jet milling and ball grinding. The chitosan or chitosan derivative employed in the present invention may be prepared by spray drying, freeze drying or by other means.

Spray drying, a one-step process that produces spherical and potentially respirable dried particles, has recently been disclosed as an alternative technique to freeze-drying for formulating gene delivery vectors for pulmonary administration. Following freeze-drying or spray-drying in the presence of a suitable protecting excipient, lipid:polycation:DNA (LPD) gene vectors retain their structural integrity. The biological functionality, i.e. in vitro gene expression efficiency, of spray-dried powders following prolonged storage at room temperature has been shown to be at least comparable to that of freshly prepared aqueous systems.

However, the respirable fraction of these formulations has, previous to the present invention, been inadequate due to aggregation of the spray-dried particles. The formulation of more dispersible, and hence more respirable, spray-dried particles would progress the development of dried powder formulations for pulmonary therapy. Use of the present invention thus permits the enhancement of the dispersibility of spray-dried particles and, hence, an improvement in their pulmonary deposition.

We have investigated the affect of modifying the surface charge and surface activity of, for example, the spray-dried product to decrease particle aggregation and increase the respirable fraction. The present invention can involve the use of the cationic polymer chitosan and its derivatives as ‘dispersibility-enhancer’ excipient in a, for example, spray-dried dry powder composition.

Chitosan and its derivatives contain a high number of positive charges which can promote particle deaggregation, and hence dispersibility, by electronic repulsion. Chitosan and its derivatives are biodegradable and have demonstrated low toxicity. In addition, chitosan and its derivatives have shown some ability in enhancing drug absorption (M. Thanou et al., Advanced Drug Delivery Reviews 52 (2001) 117-126). Chitosan is a polymer derived from arthropods such as prawn, lobster and crab.

The trimethylated chitosan chloride derivative has the structural formula:

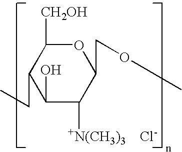

The chitosan or chitosan derivative employed in the present invention can have a molecular weight in the range of 5 kDa to 2000 kDa.

Chitosan and its derivatives are commercially available. Examples of such commercially available products include:

ChitoClear™ cosmetic grade having a molecular weight of about 100,000 and available from Primex ingredients ASA, Norway; and
Chitosan α-lipoate, Chitosan DL-6,8-thioctate (ThioMer™ 301) and Thioglycolate chitosan (Thiomer™ 321) available from Carbomer Inc., San Diego, Calif., USA.

Chitosan is composed of glucosamine and N-acetyl glucosamine linked in a (1-4) manner, the glucosamine:N-acetyl glucosamine ratio being referred to as the degree of deacetylation.

Preferred samples of chitosan have a degree of deactylation from 40% to 98%.

The chitosan derivatives employed can be esters, ether or other derivatives formed by interaction of acyl or alkyl groups with the OH groups, or amines, amides or other derivatives formed by interaction of alkyl or acyl groups with the NH₂ groups on chitosan.

Suitably, any acyl or alkyl substituents are saturated or unsaturated, branched or unbranched C₁ to C₆. A preferred alkyl group is methyl and a preferred acyl group is methacrylate. The alkyl and acyl groups can include members selected from the group comprising —CH₃, —CH₂CH₃, —CH₂CH₂CH₃, —CH(CH₃)₂, —(CH₂)₃CH₃, —CH(CH₃)CH₂CH₃, —C(CH₃)₃, —(CH₂)₄CH₃, —CH(CH₃)(CH₂)₂CH₃, —CH(CH₂CH₃)₂, —(CH₂)₅CH₃, —CH(CH₃)(CH₂)₃CH₃, —CH(CH₂CH₃)(CH₂)₂CH₃, —CH₂CH═CH₂, —CH(CH₃)CH═CH₂, —CHCH═CHCH═CH₂ and —C₆H₅.

Chitosan derivatives formed by interaction with, at least some of, its NH₂ sites on chitosan are preferred. The NH₂ sites can be substituted by one, two or three substitutents. Preferably, substituents are selected from the group comprising: C₁-C₆ alkyl groups and C₁-C₆ acyl groups. Trisubstitution of the NH₂ sites of chitosan is preferred so as to yield a cationic polymer having exceptionally good solubility characteristics. Trisubstitution of the NH₂ sites on chitosan may also play a role in enhancing its dispersability characteristics. Preferably a chitosan derivative is employed which comprises chitosan trisubstituted at from 10 to 90%, preferably at from 30 to 70%, more preferably at from 40 to 60%, of its NH₂ sites by C₁ to C₆ alkyl, preferably methyl.

Chitosan derivatives formed by interaction with its OH sites preferably have 20 to 99%, preferably from 30 to 95%, of its OH sites substituted. A preferred substituent is acyl.

Preferred chitosan derivatives that can be employed are chitosan having a degree of methacrylation of from 30% to 95% of its OH sites and chitosan having from 10 to 90% of its NH₂ sites trimethylated.

Salts of chitosan and its derivatives can also be employed. Examples of suitable salts include chlorides, nitrates, phosphates, sulphates, xanthates, hydrochlorides, lactates and acetates.

The actual size of the particles prepared by spray drying may depend on the spray drying conditions employed. We have been found, for example, that spray drying ChitoClear™ having a molecular weight of about 100,000 at an outlet temperature during spray drying of between 60 and 100° C., preferably 80 to 90° C., produced a particle size distributions of from 0.1 to 7 μm, preferably 0.5 to 5 μm, more preferably 1 to 5 μm. The initial ChitoClear™ material was dissolved in acidic solution with a pH value of less than 6, adjusted by use of an inorganic acid or an organic acid.

Preferably the present invention employs a chitosan or a chitosan derivative having a molecular weight within the range 5 kDa to 2000 kDa, more preferably within the range of from 20 kDa to 1000 kDa, even more preferably within the range 50 kDa to 500 kDa. Chitosan or chitosan derivative is preferably present in the dry powder composition at a level, with respect to the total weight of the composition, of 1 to 95 wt %, more preferably 5 to 95 wt %, more preferably 10 to 90 wt %, even more preferably 20 to 90 wt %, and even more preferably 40 to 90 wt %.

Chitosan is suitably present in a dry powder composition at a level, with respect to the total weight of the composition, of 3 wt % to 90 wt %, more suitably 6 wt % to 67 wt % and even more suitably 9 wt % to 50 wt %.

A soluble low molecular weight chitosan or a soluble chitosan derivative such as trimethylated chitosan is suitably present in a dry powder composition at a level, measured with respect to the total weight of the composition, of 1 wt % to 91 wt %, more suitable 2 wt % to 50 wt %, even more suitably 5 wt % to 9 wt %.

Where an excipient such as a sugar is present in the dry powder composition, the composition preferably comprises 0.001 to 30 wt % medicament, 1 to 50 wt %, preferably 1 to 40 wt %, chitosan or chitosan derivative and 49.999 to 98.999 wt %, preferably 59.999 to 98.999 wt %, excipient, for example sugar, measured with respect to the total weight of the composition. Disaccharide sugars are preferred as the primary excipient.

The molecular weight of the chitosan or a chitosan derivatives, and in the case of a substituted chitosan derivative the degree of substitution, are factors in determining the water solubility of the chitosan or a chitosan derivative, and hence the method of preparation of a dry powder composition containing chitosan or a chitosan derivative. Chitosan or a chitosan derivative such as, for example O-substituted chitosan, having a molecular weight less than approximately 3500 is deemed water soluble. Chitosan or a chitosan derivative such as, for example, O-substituted chitosan, having a molecular weight greater than approximately 5000 is, generally, found to be water insoluble. The solubility is also affected according to the nature of any chitosan derivative employed. For example, trimethylated chitosan chloride with a sufficiently high degree of substitution is water soluble at any molecular weight. Trimethylated chitosan chloride having at least a 50% degree of trisubstitution has been found to be water soluble.

Dry powder compositions suitable for use in the present invention can, for example, be made by any of the following methods:

(i) Mixing a particulate medicament or medicament containing composition, for example, prepared by spray drying, with chitosan, for example ChitoClear™, or chitosan derivative by a physical method such as mechanical stirring, agitation, vibration or shaking, scratching or squeezing with for example pestle and mortar or other similar tools, or through gas flow. A sugar, such as the disaccharide lactose, may be admixed with the medicament prior to spray drying. Preferably the resulting dry powder composition comprises 0.001 to 30 wt %, more preferably 0.10 to 10 wt %, medicament, 1 to 30 wt %, more preferably 10 to 20 wt %, chitosan or chitosan derivative and 69.999 to 98.999 wt %, more preferably 79.999 to 89.999 wt % disaccharide.

(ii) As method (i), except that the chitosan or a chitosan derivative had been previously spray dried. For example, the ChitoClear™ was dissolved in acidic solution with pH value less than 6 adjusted by an inorganic or an organic acid. The outlet temperature during spray drying is from 60 to 110° C., preferably 80 to 90° C. A sugar, such as the disaccharide lactose, may be admixed with the medicament prior to spray drying. Preferably the resulting dry powder composition comprises 0.001 to 30 wt %, more preferably 0.1 to 10 wt %, medicament, 1 to 40 wt %, more preferably 20 to 30 wt %, chitosan or chitosan derivative, and 59.999 to 89.999 wt %, more preferably 69.999 to 79.999 wt %, disaccharide.

(iii) Spray drying an aqueous solution containing chitosan or a chitosan derivative, a medicament compound or a medicament-containing composition, and, optionally, one or more additional excipients such as, for example, one or more sugars, in particular disaccharides, for example, lactose. Preferably the resulting dry powder composition comprises 0.001 to 30 wt %, more preferably 0.01 to 10 wt %, medicament, 1 to 20 wt %, more preferably 5 to 10 wt %, chitosan or chitosan derivative, and 79.999 to 98.999 wt %, more preferably 89.999 to 94.999 wt %, disaccharide.

(iv) As method (iii), with the additional step that the resultant spray dried chitosan or a chitosan derivative and medicament-containing product is admixed with further chitosan or a chitosan derivative, which may or may not have been previously spray dried. Preferably the resulting dry powder composition comprises 0.001 to 30 wt %, more preferably 0.01 to 10 wt %, medicament, 1 to 40 wt %, more preferably 30 to 40 wt %, chitosan or chitosan derivative, and 59.999 to 98.999 wt %, more preferably 59.999 to 69.999 wt % disaccharide.

Suitably, a dry powder composition for use in the present invention comprises a weight ratio of chitosan to medicament or to medicament-containing particles within the range of chitosan to medicament or to medicament-containing particles of from 1:1000 to 1:0.001, preferably within the range 1:100 to 1:0.01, more preferably within the range of from 1:10 to 1:0.1.

Suitably, a dry powder composition for use in the present invention comprises a weight ratio of a chitosan derivative, such as chitosan substituted at, at least some of, its NH₂ sites by one, two or three members selected from the group comprising C₁-C₆ alkyl and C₁-C₆ acyl, for example, trimethylated chitosan, comprises a weight ratio of chitosan derivative to medicament or to medicament-containing particles within the range of from 100:1 to 1:0.001, preferably within the range of from 1:50 to 1:0.5, more preferably within the range of from 1:20 to 1:1.

The present dry powder compositions comprise particles in a dry state, by which is meant a particulate material that feels dry to touch and flows as a powder.

The medicament can be any medicament that can usefully be delivered in the form of a dry powder composition.

Medicaments appropriate for delivery in the form of a dry powder composition intended for use as in a dry powder inhaler include medicaments for use in the treatment and prevention of asthma and other conditions associated with reversible airways obstruction. Such medicament either alone or in any combination can be selected from the group comprising:
(i) salbutamol, salbutamol sulphate, mixtures thereof and physiologically acceptable salts and solvates thereof;
(ii) terbutaline, terbutaline sulphate, mixtures thereof and physiologically acceptable salts and solvates thereof;
(iii) beclomethasone diproprionate and physiologically acceptable solvates thereof;
(iv) budesonide and physiologically acceptable solvates thereof;
(v) triamcinolone acetonide and physiologically acceptable solvates thereof;
(vi) ipratropium bromide and physiologically acceptable salts and solvates thereof;
(vii) corticosteroid or bronchodilator; and
(viii) leukotriene antagonists.

Other examples of particulate medicaments suitable for oral or nasal inhalation so as to achieve pulmonary delivery by means of the present invention include:
(ix) peptides, proteins, nucleic acids and derivatives thereof for use in the treatment and prevention of disease states; and
(x) insulin, calcitonin, growth hormone, lutenising hormone release hormone (LHRH), leuprolide, oxytocin and physiologically acceptable salts and solvates thereof for use in the treatment and prevention of disease states including diabetes.

Further examples of appropriate medicaments which can be formed into the present particulate compositions may additionally be selected from, for example, analgesics, e.g., codeine, dihydromorphine, ergotamine, fentanyl or morphine; anginal preparations, e.g., diltiazen; antiallergics, e.g., cromoglycate, ketotifen or nedocromil; anti-infectives e.g., cephalosporins, penicillins, streptomycin, sulphonamides, tetracyclines and pentamidine; antihistamines, e.g. methapyrilene; antiinflammatories, e.g., beclomethasone diproprionate, fluticasone propionate, flunisolide, budesonide, rofleponide, mometasone furoate or triamcinolone acetonide; antitussives, e.g., noscapine; bronchodilators, e.g., albuterol, salmeterol, ephedrine, adrenaline, fenoterol, formoterol, isoprenaline, metaproterenol, phenylephrine, phenylpropanolamine, pirbuterol, reproterol, rimiterol, terbutaline, isoetharine, tulobuterol, or (−)-4-amino-3,5-dichlor-α[[[6-[2-(2-pyridinyl)ethoxyl]hexyl]methyl][benzenemethanol; diuretics, e.g., amiloride; anticholinergics, e.g., ipratropium, tiotropium, atropine or oxitropium. Hormones, e.g., cortisone, hydrocortisone or prednisolone; xanthines, e.g., aminophylline, choline theophyllinate, lysine theophyllinate or theophylline; therapeutic proteins and peptides, e.g. insulin or glucagon. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g. hydrates) to optimise the activity and/or stability of the medicament.

Preferred medicaments are selected from nucleic acids, salbutamol, salmeterol, fluticasone propionate and beclomethasone dipropionate and salts or solvates thereof, e.g., the sulphate of albuterol and the xinafoate of salmeterol.

Medicaments can also be delivered in combinations. Preferred formulations containing combinations of active ingredients contain salbutamol (e.g., as the free base or the sulphate salt) or salmeterol (e.g., as the xinafoate salt) in combination with an anti-inflammatory steroid such as a beclomethasone ester (e.g., the dipropionate) or a fluticasone ester (e.g., the propionate).

Examples of nucleic acid systems include corrective plasmid DNA (PDNA) constructs capable of expressing a therapeutic gene. Preferred nucleic acid systems are pDNA constructs whose stability and activity have been enhanced by pre-condensation with a polycationic peptide, for example, a protamine such as protamine sulphate. Suitably any protamine is included at a concentration of 0.1 to 10 mg/mg, more suitably 0.8 to 2 mg/mg, with respect to the nucleic acid.

The dosage requirements for any one medicament will be those conventionally employed in, for example, inhalers. For example, where the active material is salbutamol for use in relation to asthma the inhaler is employed as required, usually 1 or 2 actuations (i.e. puffs) between 0 and 4 times per day, with a single dose comprising 100 micrograms of salbutamol.

An additional material that can beneficially be included in the particles comprising the present dry powder composition is an excipient such as sugar. Examples of suitable sugars include mono and/or disacharides, such as for example lactose, sucrose, trehalose, mannitol and mixtures thereof. Disaccharides, such as for example lactose, sucrose, trehalose or mixtures thereof, are preferred. When a sugar, in particular a disaccharide sugar, is present in the present dry powder compositions, it is suitably present as a primary excipient, by which is meant it is present in an amount substantially equal to or, preferably, in an amount greater than the amount of chitosan or chitosan derivative present. The dry powder composition preferably comprises 0.001 to 30 wt %, more preferably 0.01 to 10 wt %, medicament, 1 to 50 wt %, preferably 1 to 40 wt %, chitosan or chitosan derivative and 49.999 to 98.999 wt %, preferably 59.999 to 98.999 wt %, sugar, which is preferably a disaccharide sugar. The sugar, which is preferably a disaccharide sugar, is suitably present in the form of particles having an average diameter of from 0.5 to 11 μm, more preferably 0.5 to 3.5 μm. Preferably the dry powder composition comprises particulate material wherein the particulate material comprising the medicament, and optionally the additional material, such as a disaccharide sugar, and/or the chitosan or chitosan derivative is prepared by a process selected from the group comprising spray drying, freeze drying and mechanical techniques, preferably jet milling and ball milling. Spray drying is preferred. Such processes where the medicament and any additional material are prepared together, for example by spray drying a solution or suspension containing the medicament and the additional material such as a disaccharide sugar, can result in particles comprising an admixture, for example in the form of a matrix, of the medicament and additional material, such as a disaccharide sugar, having a diameter of from 0.5 to 11 μm, preferably 0.5 to 3.5 μm.

The inclusion of a sugar, in particular a disaccharide, in the present particles can confer stability on the active material during processing, for example spray drying, and storage of the particulate composition. Examples of active materials whose stability may be increased in the presence of sugar, in particular a disaccharide, include nucleic acid, peptide and/or protein based drugs. Lactose and sucrose are particularly preferred for use with nucleic acids. Spray drying nucleic acids in a sugar solution of, for example, lactose or sucrose protects and stabilises the active material.

The weight ratio of the medicament to any sugar present in the particles preferably lies within the range of from 99:1 to 1:99, more preferably from 99:1 to about 20:80.

Additional excipients that may be included in the present dry powder compositions, either with or without any sugar present, include one or members selected from the group comprising dimethyl-β-cyclodextrin, sodium taurocholate, carnitine hydrochloride and an amino acid such as leucine, arginine, aspartic acid, threonine and phenylalanine.

The chitosan or chitosan derivative is believed to act as a dispersibility enhancer through modification of the surface morphology of the excipient or carrier, such as a disaccharide sugar.

Further examples of additional materials that can beneficially be included in the present dry powder composition, particularly when the active material is a nucleic acid, include one or more cationic lipids as they may facilitate cellular entry of genetic material and a peptide to protect the nucleic acid. An example of a suitable cationic lipid is 1,2-dioleoyl-3-trimethylammonium propane (DOTAP).

Any additional material present is suitably in the form of a matrix incorporating the medicament. Thus, by “medicament-containing particles” is meant particles comprising solely medicament, particles comprising medicament in combination with chitosan or chitosan derivative, particles comprising medicament in combination with additional material such as a disaccharide sugar or particles comprising medicament, chitosan or chitosan derivative and additional material such as a disaccharide sugar. Medicament-containing particles preferably have a diameter of 0.5 to 11 μm, preferably 0.5 to 3.5 μm. Such medicament-containing particles comprising, for example medicament and an additional material, such as a disaccharide sugar, can be prepared by, for example, by spray drying a solution or a suspension comprising the medicament and the additional material, such as a disaccharide sugar.

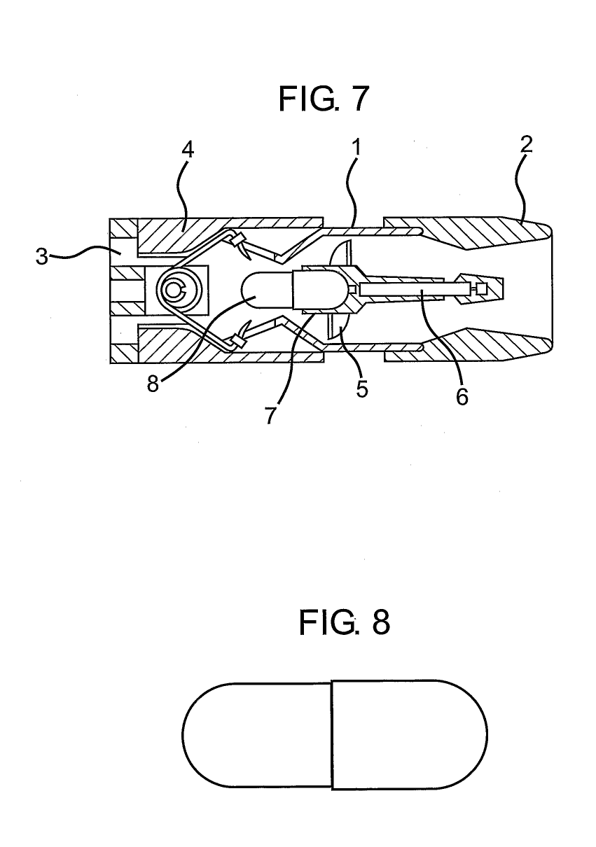

The use of the present invention is suitably implemented by means of a dry powder inhaler. Dry powder inhalers are known devices. Accompanying FIG. 7 illustrates, in cross section, a dry powder inhaler of the type known as a Spinhaler™.

The Spinhaler™ consists of a tubular body (1) threaded at one end to receive the mouthpiece (2), and with an air inlet (3) at the other end. The body (1) is fitted with an external sleeve (4) which can slide from its normal position against the air inlet (3), to the mouthpiece (2) and back again. The body (1) unscrews from the mouthpiece (2). A propeller (5), inside the tubular body (1), is rotatable on a stainless steel spindle (6). The propeller (5) has a slotted cup (7) into which a capsule (8) can be fitted.

In use the movement of the inspired air causes the propeller (5) to rotate and vibrate at high speed. When a powder filled capsule (8) is fitted in the device, its envelope is perforated by moving the external sleeve (4) of the Spinhaler™. During deep and rapid inspiration through the device the contents of the capsule (8) are dispersed into the inspired air by the action of the propeller (5). In this way, the drug particles can be carried deep into the lungs to the site where their action is required.

Loading and Inhaling the Dose:

1. To prepare the Spinhaler™ for use, the appliance is held vertically with the mouthpiece downwards and the body is unscrewed from the mouthpiece.

2. A capsule is inserted firmly into the cup on the propeller. Check that the propeller rotates freely.

3. The mouthpiece is screwed back into position.

4. Whilst still holding the Spinhaler™ vertically (mouthpiece downwards), the external sleeve is forced downwards as far as it will go and then back to its original position. (This step may be repeated a second time for optimal piercing).

5. The capsule has now been pierced and the appliance is ready for use.

Accompanying FIG. 8 illustrates a container in the form of an individual capsule containing the dry powder composition adapted for use in a dry powder inhaler.

Embodiments of the present invention will now be described by way of example only with reference to the accompanying drawings:

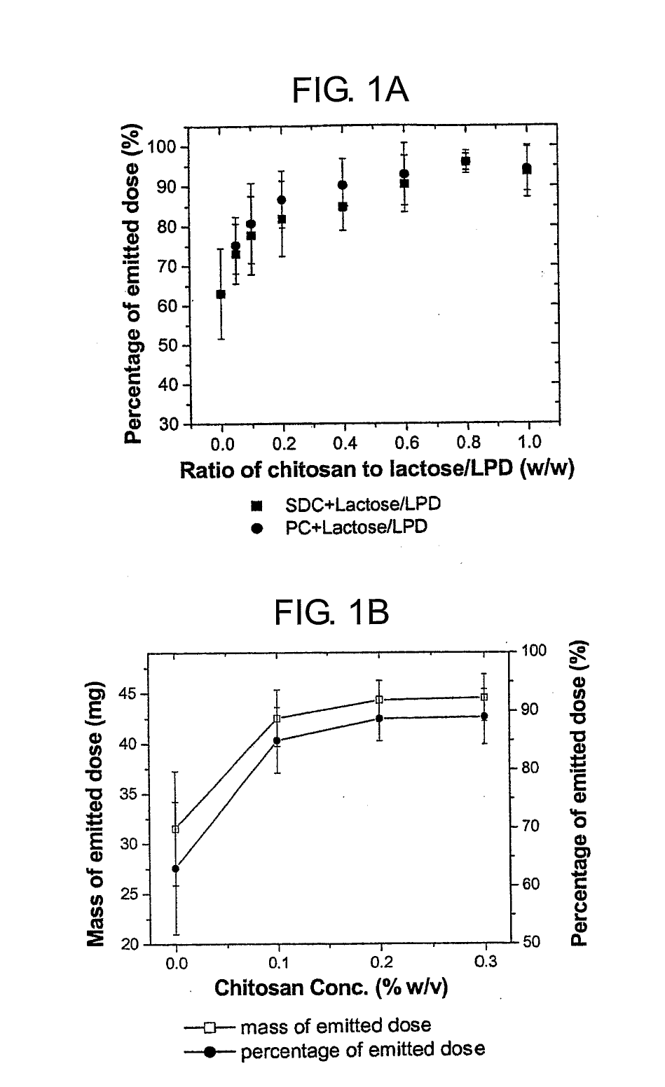

FIGS. 1A and 1B show two graphs that illustrate the percentage of composition that is emitted from a dry powder inhaler capsule as a function of concentration of, respectively, post-mixed chitosan or spray dried chitosan (FIG. 1A) and soluble chitosan added before spray drying (FIG. 1B);

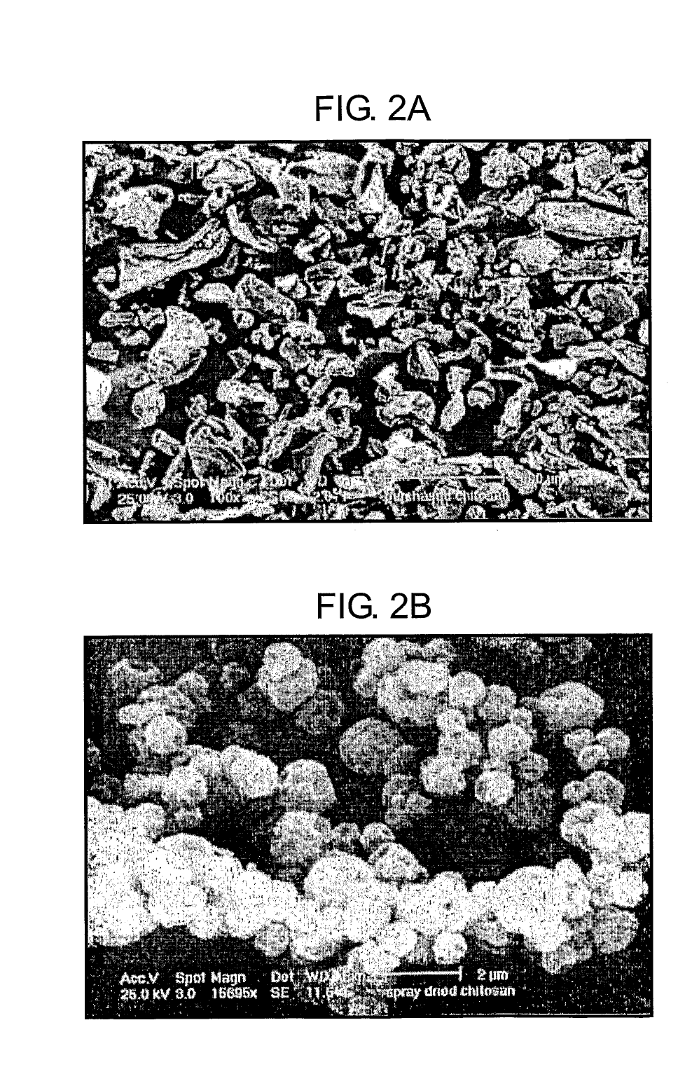

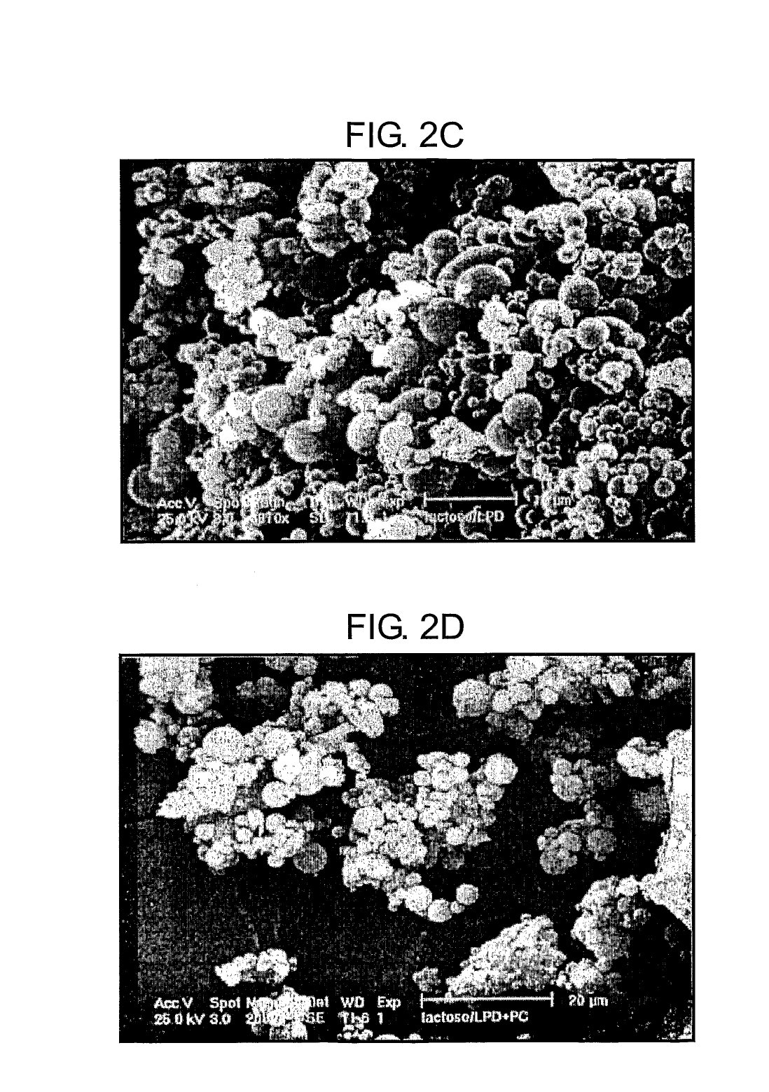

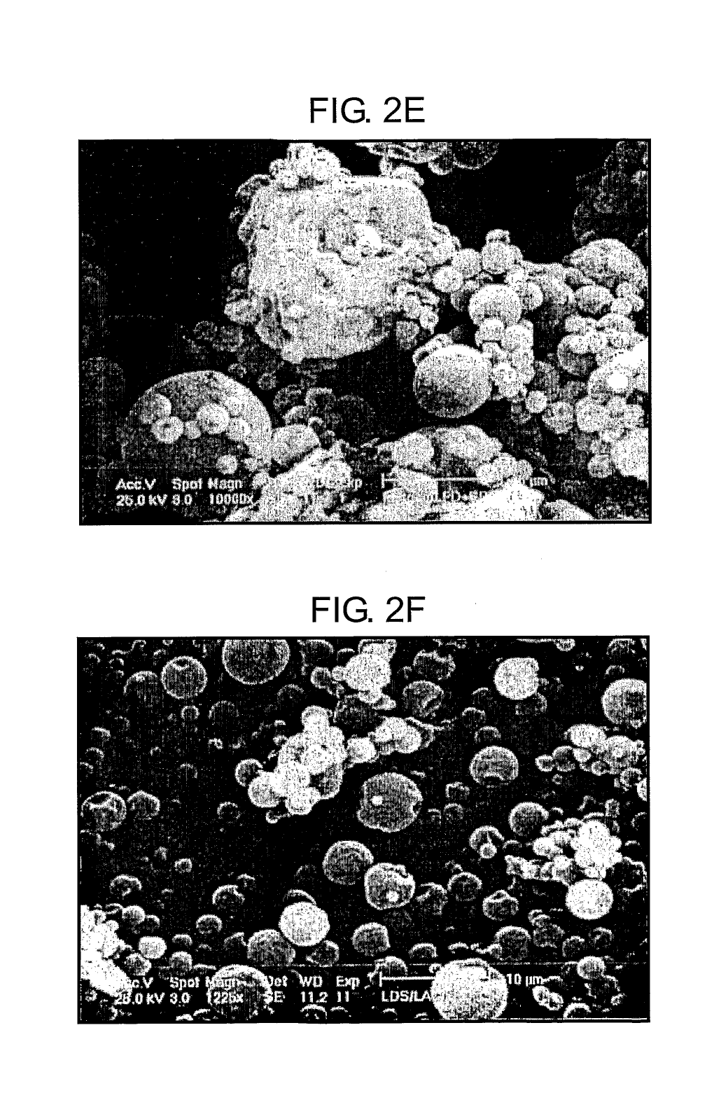

FIGS. 2A to 2F show scanning electron microscopy of, respectively, chitosan (FIG. 2A), spray-dried chitosan (FIG. 2B), lactose/LPD particles (FIG. 2C), lactose/LPD particles post-mixed with high MW chitosan (FIG. 2D), lactose/LPD particles post-mixed with high MW spray-dried chitosan (FIG. 2E), and spray-dried lactose/low MW chitosan derivative/LPD particles (FIG. 2F);

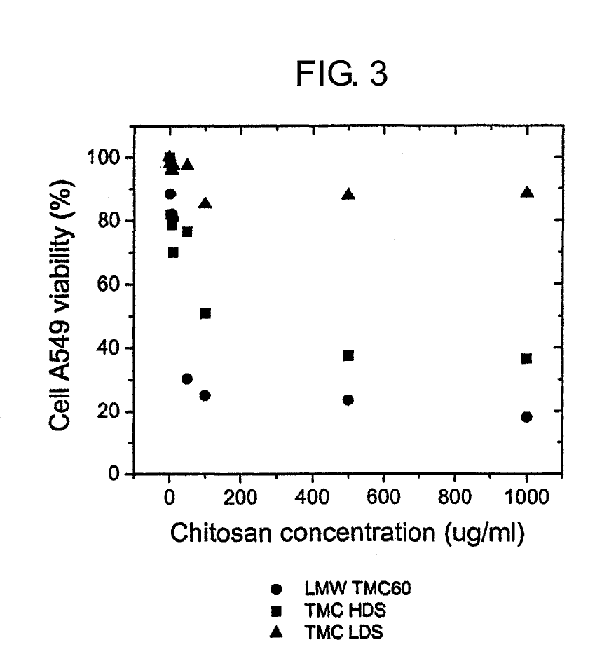

FIG. 3 illustrates the MTT assay of three soluble chitosan derivatives exposed to a human lung bronchiocarcinoma cell line (A549 cells) in culture;

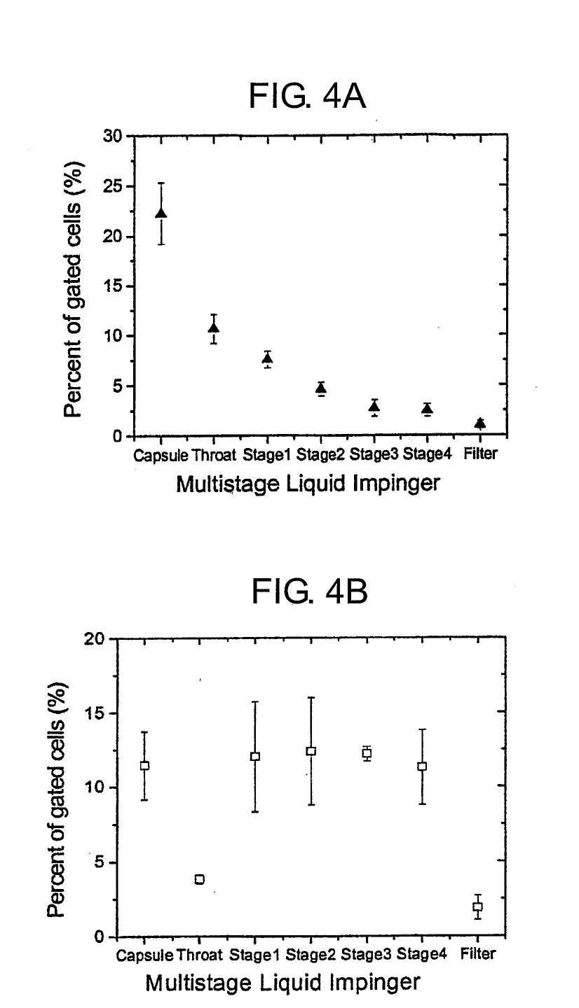

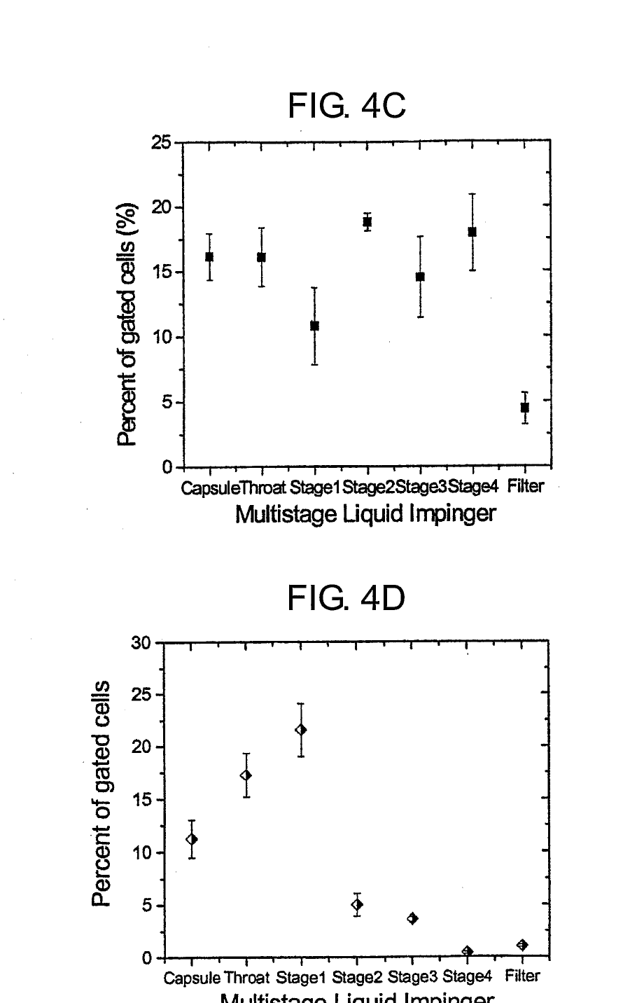

FIGS. 4A to 4D show four graphs illustrating the percentage of cells on each stage of an MSLI showing fluorescence attributed to the expression of a fluorescent gene product with respect to, respectively, lactose/LPD (FIG. 4A), lactose/LPD post-mixed with chitosan (FIG. 4B), lactose/LPD post/mixed with spray-dried chitosan (FIG. 4C), and spray-dried lactose/LPD/chitosan derivative (FIG. 4D);

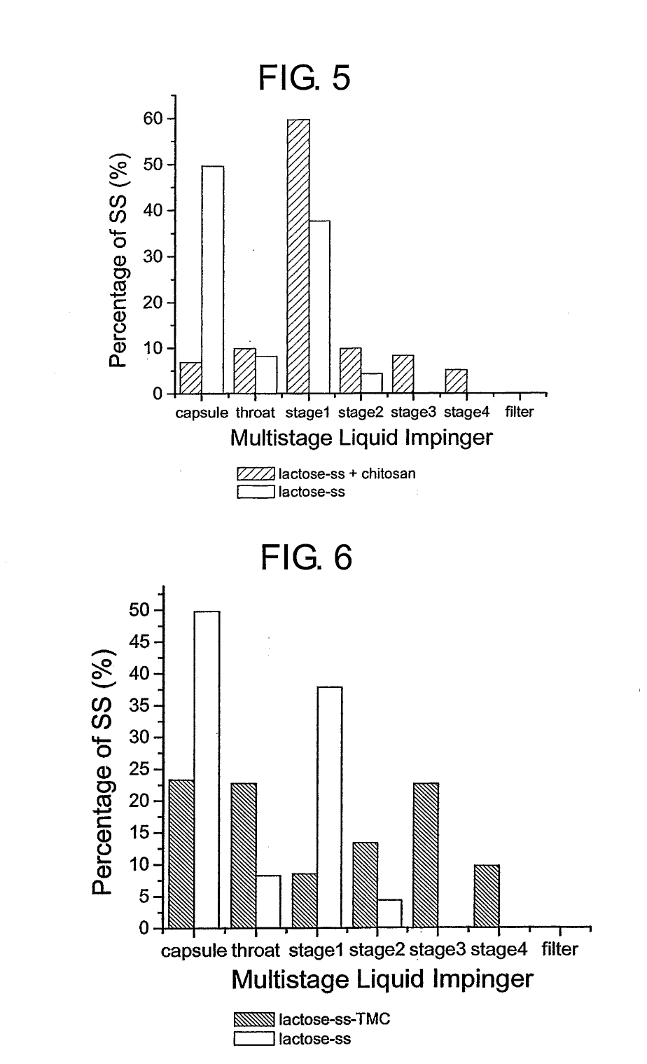

FIG. 5 shows a bar chart illustrating the dispersibility of lactose-salbutamol sulphate particles both with and without chitosan; and

FIG. 6 shows a bar chart illustrating the dispersibility of lactose-salbutamol sulphate particles both with and without a chitosan derivative.

### METHODOLOGY

Preparation of Dry Powders

Lipid:polycation:DNA (LPD) complex preparations, typically comprising pDNA 400 μg, protamine 800 μg and lipid 1200 μg in 50 ml 3% lactose solution, were spray-dried (Büchi B-191 Mini Spray-dryer) to produce a dry powder. The operating conditions employed were: inlet temperature (temperature of drying air) 150° C.; spray flow rate (quantity of pressurized air necessary for spraying the solution) 600 L/hr, aspirator setting (throughput of drying air) 35 m³/hr, pump setting 450 ml/r. These conditions resulted in an outlet temperature (temperature of air stream containing solid particles) of 80-85° C.

A range of examples of dry powder compositions including chitosan or chitosan derivatives was prepared that varied in their composition as well as in their method of preparation.

In some examples chitosan, or a chitosan derivative, was added to the lactose/LPD suspension prior to spray-drying. In other examples chitosan or spray-dried chitosan was post-mixed (for 1 minute in a pestle and mortar) with the spray-dried lactose/LPD powder.

Yield of Dry Powders Following Spray-Drying

The yield of the dry powders following spray-drying is shown in Table 1.

TABLE 1
The percentage recovery (yield) of the spray-dried product

| | Approximate |
|---|---|
| Spray-Dried Formulation | Yield (%) |
| Lactose/LPD | 35 |
| Chitosan | 60 |
| Lactose/Chitosan/LPD | 55 |
| Lactose/Chitosan derivative/LPD | 55 |
| Lactose/Chitosan derivative/salbutamol sulphate | 63 |

It was observed that for powders prepared with the addition of chitosan or a chitosan derivative the powder was mainly present in the lower chamber of the spray dryer and not dispersed throughout the apparatus as is the case with lactose alone.

Emitted Dose

For a range of samples, 50 mg of dried powder was loaded into pre-punctured size 2 gelatin capsules and fired from a dry powder inhaler (Spinhaler) at 60 L/min flow rate, 35% relative humidity, 20° C. The emitted dose was calculated as the difference in weight between the pre-punctured capsule before and following firing the inhaler (FIG. 1). The data displayed in FIG. 1 show that the amount of actual dose emitted from the inhaler relative to the intended dose increases, up to a plateau, with the increase in the amount of chitosan present in the dry powder formulation. The chitosan employed had a molecular weight of 100 kDa and an average particle size of 1.7 μm.

Both high MW chitosan (PC) and spray-dried high MW chitosan (SDC) were found to enhance the emission of the powder from the capsule when post-mixed with spray-dried lactose/LPD powder. The emitted dose is also enhanced when a soluble N-trisubstituted chitosan derivative is included in the formulation before spray-drying. Each of the chitosans, as well as the N-trisubstituted chitosan, employed in these examples had a molecular weight of 100 kDa.

Fine Particle Fraction

The fine particle fraction of the DNA containing dry powders was determined as follows:

(i) Fine particle fraction (<6.8 μm): The gene expression mediated by particles delivered to stage 3, stage 4 and the filter of the multistage liquid impinger (i.e. particles with a diameter less than 6.8 μm) divided by the total gene expression at stages 1, 2, 3 and 4 and filter and device throat and powder remaining in the capsule after inhaler firing. Flow rate: 60 litres/min.

(ii) Fine particle fraction (<3.1 μm): The gene expression mediated by particles delivered to stage 4 and the filter of the multistage liquid impinger (i.e. particles with a diameter less than <3.1 μm) divided by the total gene expression at stages 1, 2, 3, 4 and filter and device throat and powder remaining in the capsule after inhaler firing. Flow rate 60 litres/min.

For non-nucleic acid medicaments the fine particle fraction of a dry powder composition is determined by dividing the weight of particles delivered to stage 3, stage 4 and the filter of the multistage liquid impinger (i.e. particles with a diameter less than 3.1 μm) divided by the total weight of dry powder composition at stages 1, 2, 3, 4 and filter and device throat and powder remaining in the capsule after the inhaler firing. Flow rate: 60 litres/min.

The fine particle fraction is thus the separable fraction of a dry powder composition that is capable of being delivered deep within the lungs through the lower airways to the mucosal surface of the alveoli.

We have observed the following fine particle fractions:

Composition 1:

Spray-dried lactose incorporating an LPD (lipid:polycation:pDNA) complex comprising 1,2-Dioleoyl-3-Trimethylammonium-Propane (DOTAP), protamine sulphate and pEGFP—N1 plasmid DNA post-mixed with chitosan (ChitoClear™ cosmetic grade. MW˜100,000, Primex Ingredients ASA, Norway). The weight ratio of chitosan to lactose was 1:5. The average particle size of the lactose was 3.8 μm and the average particle size of the chitosan was 63 μm.

Fine Particle Fraction: <6.8 μm: 38.5 wt %; <3.1 μm: 19.7 wt %

Composition 2: Spray-dried lactose incorporating an LPD (lipid-polycation:pDNA) complex comprising 1,2-Dioleoyl-3-Trimethylammonium-Propane (DOTAP), protamine sulphate and pEGFP-N1 plasmid DNA post-mixed with spray-dried chitosan (ChitoClear™ cosmetic grade. MW˜1000,000, Primex Ingredients ASA, Norway). The weight ratio of chitosan to lactose was 2:5. The average particle size of the chitosan was 1.3 μm. The average particle size of the lactose was 3.8 μm.

Fine Particle Fraction: <6.8 μm: 38.3 wt %; <3.1 μm: 22.6 wt %

Composition 3: Spray-dried lactose incorporating an LPD (lipid-polycation:pDNA) complex comprising 1,2-Dioleoyl-3-Trimethylammonium-Propane (DOTAP), protamine sulphate and pEGFP-N1 plasmid DNA and further incorporating a chitosan derivative, 51% N-trimethylated chitosan (TM-chitosan; MW˜100,000), in the spray-drying solution. The weight ratio of the chitosan derivative to the lactose was 1:10. The average particle size of the lactose/chitosan derivative complex was 3.3 μm.

Fine Particle Fraction: <6.8 μm: 9.4 wt %; <3.1 μm: 3.4 wt %

Composition 4: Spray-dried lactose incorporating an LPD (lipid:polycation:pDNA) complex comprising 1,2-Dioleoyl-3-Trimethylammonium-Propane (DOTAP), protamine sulphate and pEGFP-N1 plasmid DNA and further incorporating a chitosan derivative, 51% N-trimethylated chitosan (TM-chitosan; MW˜100,000), in the spray-drying solution post-mixed with chitosan (ChitoClear™ cosmetic grade. MW˜100,000, Primex Ingredients ASA, Norway). The weight ratio of the chitosan derivative to lactose in the co-spray dried composition was 1:10. The weight ratio of the co-spray dried composition postmixed with the chitosan was 5:2. The particle size of the co-sprayed dried material was 3.3 μm. The particle size of the postmixed chitosan was 63 μm.

Fine Particle Fraction: <6.8 μm: 20.7 wt %; <3.1 μm: 16.6 wt %

Respirable Particle Fraction

### EXAMPLE 1

A solution of lactose and salbutamol sulphate was spray dried, with the outlet temperature of the spray dryer at a temperature of 83 to 85° C., so as to yield a spray dried powder comprising 1.5 g lactose and 4 mg salbutamol sulphate. 500 mg of the spray dried lactose-salbutamol sulphate powder was loaded in 50 mg doses into 10 gelatin capsules.

500 mg of the spray dried lactose-salbutamol powder was admixed with 200 mg insoluble chitosan (molecular weight 100,000) using a pestle and mortar. 700 mg of this mixture was loaded into 14 gelatin capsules to provide 50 mg doses.

The multi-stage liquid impinger (MSLI) was used for in-vitro deposition at a flow rate of 60 L/min, 20° C. and relative humidity 35%. The particle size cut-off diameter under these conditions was 13, 6.8, 3.1 and 1.7 μm, respectively, from stage 1 to stage 4. 20 ml of deionized water was contained in each stage during deposition for collecting the deposited powder. After deposition, the solution in each stage was collected for the analysis of salbutamol sulphate. Washings from the capsule, inhaler, throat and MSLI filter were also collected and analysed.

Salbutamol concentration was determined using reverse-phase HPLC with a C18 column at 278 nm. The mobile phase (flow rate=1 ml/min) was methanol-water at a volume ratio of 55:45 (containing heptane sulphonic acid 1.1013 g/L), whose pH value was adjusted to 3 with glacial acetic acid. Samples were prepared in internal standard solution (ethanol 600 ml, water to 1000 ml, bamethane 7 μg/ml).

FIG. 5 shows that the dispersibility of salbutamol sulphate-lactose powder was increased when it was mixed with chitosan. In the absence of chitosan, 50 wt % of the lactose-salbutamol sulphate powder remained in the capsule. In the presence of chitosan, the chitosan delivered the majority of salbutamol sulphate-lactose powder into at least stage 1, with some further deposition observed in the lower stages. Insoluble chitosan is thus seen to enhance the release from a container and to increase the dispersibility in air of spray dried salbutamol sulphate-lactose powder.

Stage 1 of the MSLI permits particles having an average diameter of less than 13 μm to pass. The results as shown in FIG. 5 indicate that use of chitosan would enhance the respirable fraction of salbutamol sulphate so as to be available, in use, to deposit in at least the upper airways of the lungs of a patient.

### EXAMPLE 2

50 ml of a solution containing salbutamol sulphate (4 mg), lactose (1.5 g) and 60% N-trimethylated chitosan chloride were spray dried using the spray drying conditions set out under Example 1. The yield of the powder was 63 wt %.

The spray dried powder was loaded in 50 mg doses into 11 gelatin capsules.

The multi-stage liquid impinger was used to measure the dispersibility of the powder as described under Example 1. The salbutamol deposition at each stage was measured using the HPLC method described under Example 1.

FIG. 6 shows that the dispersibility of the salbutamol sulphate-lactose powder was substantially increased in the presence of the N-trisubstituted chitosan derivative. In the absence of the chitosan derivative, 50 wt % of the powder remained in the capsule. In the presence of the N-trisubstituted chitosan, the emitted dose was 76.7 wt %. Deposition of the salbutamol sulphate-lactose powder in the lower stages provided a fine particle fraction of 32.9 wt % (<6.8 μm) and 9.7 wt % (<3.1 μm). Soluble chitosan derivative is thus seen to enhance the release from a container and to increase the dispersibility in air of the spray dried salbutamol sulphate-lactose powder.

Scanning Electron Microscopy

The surface characteristics of the materials has been studied using Scanning Electron Microscopy (SEM).

SEM shows that when spray-dried lactose particles are post-mixed with chitosan or spray-dried chitosan the insoluble high molecular weight chitosan appears to be surface-coated with spherical lactose particles (FIG. 2D, E). When lactose is spray-dried in the presence of soluble chitosan derivative, the chitosan does not appear as a particulate but appears to alter the surface structure of the lactose particles (FIG. 2F).

Cellular Toxicity

Prior to transfection studies the toxicity of three of the soluble chitosan derivatives was measured using a standard MTT toxicity assay.

FIG. 3 shows that only one of the chitosan derivatives (TMC LDS) was non-toxic to A549 cells in vitro. This chitosan derivative was therefore selected for biological functionality studies.

Deposition and Biological Functionality of Formulations

The ability of the formulations to deliver DNA to different areas of the lung was estimated using an artificial in vitro lung model.

A549 cells were cultured in 24-well format with media comprising DMEM, 10% foetal bovine serum, and the antibiotics penicillin and streptomycin. Cells were grown to 85% confluency at 37° C. in a humid atmosphere at 95% air/5% CO₂. A defined number of capsules each containing a total of 500 mg of powder and 133 μg of DNA (pEGFP-N1) were placed in a Spinhaler and fired at 60 L/min into a multistage liquid impinger (MSLI). The material at each stage of the MSLI was collected and 4 ml placed in each of 4 wells of a 6-well cell culture plate containing the A549 cells. The plates were incubated at 37° C. for 6 hr then surface rinsed thoroughly with PBS and fed with culture medium. The cells were returned to the incubator for a further 42 hr to allow intracellular expression of the plasmid to proceed. Subsequently, transfected cells were surface-washed with PBS, trysinised with and resuspended in 0.6 ml culture media. The percentage of cells showing Green Fluorescent Protein (GFP) associated fluorescence was quantified by flow cytometry.

FIG. 4 shows that soluble chitosan derivatives are able to increase the amount of DNA present in the throat and stage 1 (i.e. upper airway) of the MSLI. When chitosan or spray-dried chitosan is post-mixed with spray-dried lactose/LPD powder the amount of DNA in stages 3 and 4 (i.e. central and lower airway) is also increased.

In summary, we have shown that both insoluble chitosan and soluble chitosan derivatives can modify the release, dispersibility, deposition and functionality of spray-dried material. Chitosan may also benefit formulations for lung administration through its ability to enhance absorption in the pulmonary epithelia and facilitate the transit of genetic material into cells.

1. Use of chitosan or a chitosan derivative for improving the release from a container of a dry powder composition containing a medicament and/or the dispersibility in air of a dry powder composition containing a medicament.

2. Use of chitosan or a chitosan derivative in the manufacture of a dry powder composition containing a medicament for improving the delivery of the medicament to the lungs of a patient in need thereof.

3. Use according to any one of claims 1 to 2 wherein the dry powder composition comprises medicament-containing particles wherein the said medicament-containing particles have an average diameter of from 0.5 to 11 μm.

4. Use according to any one of claims 1 to 3 wherein the dry powder composition has, or medicament-containing particles in the dry powder composition have, been prepared by a process selected from the group comprising spray drying; freeze drying; and mechanical techniques, preferably jet milling and ball grinding.

5. Use according to any one of claims 1 to 4 wherein the dry powder composition comprises medicament-containing particles and the chitosan or the chitosan derivative and is administered from a dry powder inhaler so as to deliver at least the medicament-containing particles to the lungs of a patient in need thereof.

6. Use according to claim 5 wherein at least 70 wt % of an intended dose is emitted from the dry powder inhaler.

7. Use according to claim 5 or claim 6 wherein the dry powder composition yields a separable fraction of at least 5 wt %, preferably at least 10 wt %, more preferably at least 20 wt %, measured with respect to the total weight of an intended dose, the said separable fraction comprising dispersed particles having an average diameter of not more than 3.5 μm.

8. Use according to any one of claims 1 to 7 wherein the chitosan derivative comprises chitosan substituted at, at least some of, its NH₂ sites by one, two or three members selected from the group comprising: C₁-C₆ alkyl groups and C₁-C₆ acyl groups.

9. Use according to claim 8 wherein the chitosan derivative comprises chitosan trisubstituted at from 10 to 90%, preferably at from 30 to 70%, more preferably at from 40 to 60%, of its NH₂ sites by C₁-C₆ alkyl groups, preferably methyl.

10. Use according to any one of claims 1 to 9 wherein the dry powder composition comprises a chitosan derivative and a medicament or medicament-containing particles in a weight ratio of chitosan derivative to medicament or medicament-containing particles within the range 100:1 to 1:0.001, preferably within the range 1:50 to 1:0.5, more preferably within the range of from 1:20 to 1:1.

11. Use according to any one of claims 1 to 7 wherein the dry powder composition comprises chitosan and a medicament or medicament-containing particles in a weight ratio of chitosan to medicament or to medicament-containing particles within the range of from 1:1000 to 1:0.001, preferably from 1:100 to 1:0.01, more preferably from 1:10 to 1:0.1.

12. Use according to any one of claims 1 to 11 wherein the chitosan or chitosan derivative is water soluble and/or contains no cross linking formed by a cross linking agent.

13. Use according to any one of claims 1 to 12 wherein the medicament compound or medicament-containing particles is selected, either alone or in any combination, from the group comprising:
(i) salbutamol, salbutamol sulphate, mixtures thereof and physiologically acceptable salts and solvates thereof;
(ii) terbutaline, terbutaline sulphate, mixtures thereof and physiologically acceptable salts and solvates thereof;
(iii) beclomethasone diproprionate and physiologically acceptable solvates thereof;
(iv) budesonide and physiologically acceptable solvates thereof;
(v) triamcinolone acetonide and physiologically acceptable solvates thereof;
(vi) ipratropium bromide and physiologically acceptable salts and solvates thereof;
(vii) corticosteroid or bronchodilator;
(viii) leukotriene antagonists;
(ix) peptides, proteins, nucleic acids and derivatives thereof for use in the treatment and prevention of disease states; and
(x) insulin, calcitonin, growth hormone, lutenising hormone release hormone (LHRH), leuprolide, oxytocin and physiologically acceptable salts and solvates thereof for use in the treatment and prevention of disease states including diabetes.

14. Use according to any one of claims 1 to 13 wherein the dry powder composition contains sugar, preferably a sugar selected from the group comprising lactose, sucrose, trehalose and mannitol.

15. A method for delivering a medicament to the lungs of a patient in need thereof comprising administering the medicament in the form of a dry powder composition containing additionally chitosan or a chitosan derivative, the chitosan or chitosan derivative being present to enhance the release from a container of the dry powder composition and/or the dispersibility in air of the dry powder composition.

16. A method according to claim 15 wherein the dry powder composition comprising chitosan or a chitosan derivative and medicament or medicament-containing particles has any of the features set out in any one of claims 3 to 14.

17. A dry powder composition comprising 0.001 to 30 wt % medicament, 1 to 50 wt %, preferably 1 to 40 wt %, chitosan or chitosan derivative and 49.999 to 98.999 wt %, preferably 59.999 to 98.999 wt %, disaccharide, in which particulate material comprising at least the medicament comprises particles having an average diameter of from 0.5 to 11 μm.

18. A dry powder composition according to claim 17 wherein the disaccharide is selected from the group comprising lactose, sucrose and trehalose.

19. A dry powder composition according to claim 17 or claim 18 wherein particulate material comprising the medicament, and optionally the disaccharide and/or the chitosan or chitosan derivative, has been prepared by a process selected from the group comprising spray drying, freeze drying and mechanical techniques, preferably jet milling and ball grinding.

20. A dry powder composition according to any one of claims 17 to 19 wherein the medicament and/or the chitosan or chitosan derivative have any of the features set out in claims 8 to 13.

21. A container adapted for use with a dry powder inhaler, the container containing a dry powder composition according to any one of claims 17 to 20.

22. A container adapted for use with a dry powder inhaler, the container containing a dry powder composition comprising a particulate medicament or medicament-containing particles and a chitosan derivative, the chitosan derivative comprising chitosan substituted at, at least some of, its NH₂ sites by one, two or three members selected from the group comprising C₁ to C₆ alkyl groups and C₁ to C₆ acyl groups.

23. A container according to claim 22 wherein the chitosan derivative comprises chitosan trisubstituted at 10 to 90%, preferably 30 to 70%, more preferably 40 to 60%, of its NH₂ sites by C₁-C₆ alkyl, preferably methyl.

24. A container adapted for use with a dry powder inhaler, the container containing a dry powder composition comprising a particulate medicament or medicament-containing particles and chitosan or a chitosan derivative, wherein the chitosan or chitosan derivative is water soluble and/or contains no cross linking formed with a cross linking agent and comprises particles having an average diameter of more than 1 μm.

25. A container adapted for use with a dry powder inhaler, the container containing a dry powder composition comprising a particulate medicament or medicament-containing particles and chitosan or a chitosan derivative, wherein the chitosan or chitosan derivative comprises particles having an average diameter of more than 3.5 μm.

26. A container according to any one of claims 22 to 25 wherein the dry powder composition comprising the particulate medicament or medicament-containing particles and the chitosan or chitosan derivative has any of the features set out in any of claims 3 to 14.

27. A container according to any one of claims 21 to 26 wherein the container is in the form of a capsule, a blister pack or a reservoir.

28. A dry powder inhaler adapted for administering a particulate medicament or medicament-containing particles to the lungs of a patient in need thereof incorporating a container containing a dry powder composition comprising the particulate medicament or medicament-containing particles and a chitosan or a chitosan derivative, the container being according to any one of claims 21 to 27.
